(19) United States
(12) Patent Application Publication
(10) Pub. No.: US 2023/0158082 A1
(43) Pub. Date: May 25, 2023

(54) NANOVESICLES DERIVED FROM COPROCOCCUS SP. BACTERIA, AND USE THEREOF

(71) Applicant: MD HEALTHCARE INC., Seoul (KR)

(72) Inventor: Yoon-Keun Kim, Gyeonggi-do (KR)

(21) Appl. No.: 16/977,692

(22) PCT Filed: Mar. 5, 2019
(86) PCT No.: PCT/KR2019/002514

§ 371 Date: Sep. 2, 2020

(30) Foreign Application Priority Data
Mar. 6, 2018 (KR) 10-2018-0026147
Mar. 4, 2019 (KR) 10-2019-0024704

(51) Int. Cl. A61K 35/74; C12Q 1/689

(52) U.S. Cl. CPC A61K 35/74; C12Q 1/689; C12Q 1/6851

(57) ABSTRACT

The present invention relates to vesicles derived from bacteria of the genus Coprococcus and a use thereof, and the inventors experimentally confirmed that the vesicles were significantly reduced in clinical samples obtained from patients with breast cancer, ovarian cancer, bladder cancer, myocardial infarction, cardiomyopathy and atrial fibrillation, compared with a normal individual, and that when vesicles isolated from the strain were administered, the secretion of inflammatory mediators caused by pathogenic vesicles, such as E. coli-derived vesicles, was significantly inhibited. Therefore, it is expected that the vesicles derived from bacteria of the genus Coprococcus according to the present invention can be effectively used for a method of diagnosing breast cancer, ovarian cancer, bladder cancer, myocardial infarction, cardiomyopathy or atrial fibrillation, and for developing a composition for preventing, treating or alleviating gastritis, gastric cancer, colitis, colon cancer, breast cancer, ovarian cancer, bladder cancer, myocardial infarction, cardiomyopathy, atrial fibrillation or an inflammatory disease.

### TECHNICAL FIELD

The present invention relates to nanovesicles derived from bacteria of the genus Coprococcus and a use thereof, and more particularly, to a method of diagnosing breast cancer, ovarian cancer, bladder cancer, myocardial infarction, cardiomyopathy, atrial fibrillation, and the like using the nanovesicles derived from bacteria of the genus Coprococcus, and a composition for preventing, treating or alleviating gastritis, gastric cancer, colitis, colon cancer, breast cancer, ovarian cancer, bladder cancer, myocardial infarction, cardiomyopathy, atrial fibrillation or an inflammatory disease, which comprises the nanovesicle.

### BACKGROUND ART

Since the beginning of the 21st century, acute infectious diseases recognized as epidemic diseases in the past have become less important, whereas chronic inflammatory diseases accompanied by immune dysfunction caused by disharmony between humans and microbiomes have changed disease patterns as main diseases that determine the quality of life and the human lifespan. As an intractable chronic disease in the 21st century, cancer, cardiovascular diseases, chronic lung diseases, metabolic diseases, and neuropsychiatric diseases have become a big problem for public health in the country as main diseases that determine the human lifespan and the quality of life.

It is known that the number of microorganisms coexisting in the human body has reached 100 trillion, which is 10 times more than the number of human cells, and the number of microorganism genes is more than 100 times the number of human genes. A microbiota or microbiome refers to a microbial community including bacteria, archaea and eukarya present in a given habitat.

Bacteria coexisting in our body and bacteria present in the ambient environment secrete nanometer-sized vesicles in order to exchange information on genes, low molecular compounds, proteins, and the like with other cells. The mucosa forms a physical defense membrane through which particles having a size of 200 nanometers (nm) or more cannot pass, so that bacteria coexisting in the mucosa cannot pass through the mucosa, but vesicles derived from bacteria have a size of 100 nanometers or less and are absorbed into our bodies after relatively freely passing through epithelial cells via the mucosa. Bacteria-derived vesicles that are locally secreted from bacteria are absorbed via epithelial cells of the mucous membrane to thereby induce a local inflammatory response, and the vesicles having passed through the epithelial cells are systematically absorbed via lymphatic vessels and thereby distributed in respective organs, and immune and inflammatory responses are regulated in the organs in which the vesicles are distributed. For example, vesicles derived from pathogenic gram-negative bacteria such as Escherichia coli locally cause colitis, and promote a systemic inflammatory response, and blood coagulation through a vascular endothelial inflammatory response when absorbed into blood vessels, and cause insulin resistance and diabetes when absorbed into insulin-acting muscle cells. On the other hand, vesicles derived from beneficial bacteria may control a disease by controlling immune dysfunction and metabolic dysfunction caused by pathogenic vesicles.

As immune responses to factors such as bacteria-derived vesicles, Th17 immune responses characterized by the secretion of the interleukin (hereinafter, IL)—17 cytokine occur, and IL-6 is secreted when exposed to bacteria-derived vesicles, thereby inducing Th17 immune responses. Inflammation caused by the Th17 immune response is characterized by neutrophil infiltration, and during the process by which inflammation occurs, tumor necrosis factor-alpha (hereinafter, TNF-α) secreted from inflammatory cells such as neutrocyte and macrophages plays an important role.

Inflammation is a local or systemic protective mechanism against the damage or infection of cells and tissues, and is typically caused by serial biological responses occurring as humoral mediators that constitute the immune system directly response to the damage or infection or stimulate the local or systemic effector system. Examples of a main inflammatory disease include digestive diseases such as gastritis and inflammatory enteritis, oral diseases such as periodontitis, respiratory diseases such as asthma, chronic obstructive pulmonary disease (COPD), and rhinitis, dermatological diseases such as atopic dermatitis, alopecia, and psoriasis, arthritis such as degenerative arthritis and rheumatoid arthritis; and metabolic diseases such as obesity, diabetes, and hepatic cirrhosis.

Meanwhile, bacteria of the genus Coprococcus are anaerobic gram-positive bacteria symbiotically living in the human large intestine, and produce short-chain fatty acids by fermenting a carbohydrate. However, it has not been reported that bacteria of the genus Coprococcus extracellularly secrete vesicles, and particularly, no cases of applying the vesicles to the diagnosis and treatment of intractable diseases such as cancer or cardiovascular-brain diseases have been reported.

### DISCLOSURE

### Technical Problem

As a result of conducting earnest research to solve the above conventional problems, the inventors confirmed that a content of vesicles derived from bacteria of the genus Coprococcus is significantly decreased in a sample derived from a patient with breast cancer, ovarian cancer, bladder cancer, myocardial infarction, cardiomyopathy and atrial fibrillation, compared with a normal individual, through metagenomic analysis. In addition, when vesicles were isolated from Coprococcus comes belonging to bacteria of the genus Coprococcus to treat macrophages, it was confirmed that IL-6 and TNF-α secretion caused by pathogenic vesicles was significantly inhibited. Based on this, the present invention was completed.

Thus, an object of the present invention is to provide a method of providing information for diagnosis of breast cancer, ovarian cancer, bladder cancer, myocardial infarction, cardiomyopathy or atrial fibrillation.

Further, another object of the present invention is to provide a composition for preventing, treating or alleviating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, comprising vesicles derived from bacteria of the genus Coprococcus as an active ingredient.

However, a technical problem to be achieved by the present invention is not limited to the aforementioned problems, and the other problems that are not mentioned may be clearly understood by a person skilled in the art from the following description.

### Technical Solution

To achieve the object of the present invention as described above, the present invention provides a method of providing information for diagnosing breast cancer, ovarian cancer, bladder cancer, myocardial infarction, cardiomyopathy or atrial fibrillation, the method comprising the following steps:

(a) extracting DNAs from extracellular vesicles isolated from samples of a normal individual and a subject;

(b) performing polymerase chain reaction (PCR) on the extracted DNA using a pair of primers prepared based on a gene sequence present in 16 S rDNA to obtain each PCR product; and

(c) classifying a case in which a content of vesicles derived from bacteria of the genus Coprococcus is lower than that of the normal individual sample, as breast cancer, ovarian cancer, bladder cancer, myocardial infarction, cardiomyopathy or atrial fibrillation, through quantitative analysis of the PCR product.

In addition, the present invention provides a method of diagnosing breast cancer, ovarian cancer, bladder cancer, myocardial infarction, cardiomyopathy or atrial fibrillation, the method comprising the following steps:

(a) extracting DNAs from extracellular vesicles isolated from samples of a normal individual and a subject;

(b) performing polymerase chain reaction (PCR) on the extracted DNA using a pair of primers prepared based on a gene sequence present in 16 S rDNA to obtain each PCR product; and

(c) determining a case in which a content of vesicles derived from bacteria of the genus Coprococcus is lower than that of the normal individual sample, as breast cancer, ovarian cancer, bladder cancer, myocardial infarction, cardiomyopathy or atrial fibrillation, through quantitative analysis of the PCR product.

As an embodiment of the present invention, the sample in Step (a) may be blood.

As another embodiment of the present invention, the primer pair in Step (b) may be primers of SEQ ID Nos. 1 and 2.

Further, the present invention provides a pharmaceutical composition for preventing or treating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, comprising vesicles derived from bacteria of the genus Coprococcus as an active ingredient.

Further, the present invention provides a food composition for preventing or alleviating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, comprising vesicles derived from bacteria of the genus Coprococcus as an active ingredient.

Further, the present invention provides an inhalant composition for preventing or treating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, comprising vesicles derived from bacteria of the genus Coprococcus as an active ingredient.

In one embodiment of the present invention, the inflammatory disease may be one or more selected from the group consisting of atopic dermatitis, acne, psoriasis, sinusitis, rhinitis, conjunctivitis, asthma, dermatitis, an inflammatory collagen vascular disease, glomerulonephritis, encephalitis, inflammatory enteritis, chronic obstructive pulmonary disease, sepsis, septic shock, pulmonary fibrosis, undifferentiated spondylosis, undifferentiated arthrosis, arthritis, inflammatory osteolysis, chronic inflammatory diseases caused by viral or bacterial infections, ulcerative colitis, inflammatory bowel disease, rheumatoid arthritis, reactive arthritis, osteoarthritis, scleroderma, osteoporosis, atherosclerosis, myocarditis, endocarditis, pericarditis, cystic fibrosis, Hashimoto's thyroiditis, Graves' disease, leprosy, syphilis, Lyme disease, borreliosis, neuroborreliosis, tuberculosis, sarcoidosis, lupus, lupus pernio, lupus tuberculosis, lupus nephritis, systemic lupus erythematosus, macular degeneration, uveitis, irritable bowel syndrome, Crohn's disease, Sjogren's syndrome, fibromyalgia, chronic fatigue syndrome, chronic fatigue immunodeficiency syndrome, myalgic encephalomyelitis, amyotrophic lateral sclerosis, Parkinson's disease, and multiple sclerosis.

In another embodiment of the present invention, the inflammatory disease may be a disease mediated by interleukin-6 (IL-6) or tumor necrosis factor-α (TNF-α).

Further, the present invention provides a cosmetic composition for preventing or alleviating inflammatory disease comprising vesicles derived from bacteria of the genus Coprococcus as an active ingredient.

In one embodiment of the present invention, the inflammatory disease may be one or more selected from the group consisting of atopic dermatitis, acne, and psoriasis.

Further, the present invention provides a method of preventing or treating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, the method comprising a step of administering a pharmaceutical composition comprising vesicles derived from bacteria of the genus Coprococcus as an active ingredient to a subject.

Further, the present invention provides a use of vesicles derived from bacteria of the genus Coprococcus for preventing or treating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease.

In one embodiment of the present invention, the vesicles may have an average diameter of 10 to 200 nm.

In another embodiment of the present invention, the vesicles may be secreted naturally or artificially from bacteria of the genus Coprococcus.

In another embodiment of the present invention, the vesicles derived from bacteria of the genus Coprococcus may be vesicles derived from Coprococcus comes.

### Advantageous Effects

The inventors confirmed that intestinal bacteria are not absorbed into the body through epithelial cells, but bacteria-derived vesicles are absorbed, systemically distributed and then excreted out of the body through the kidneys, liver and lungs, and by metagenomic analysis for vesicles derived from bacteria present in patients' blood, also confirmed that vesicles derived from bacteria of the genus Coprococcus, which are present in blood of patients with breast cancer, ovarian cancer, bladder cancer, myocardial infarction, cardiomyopathy and atrial fibrillation significantly decrease, compared with a normal individual. In addition, when Coprococcus comes, which is one species of bacteria of the genus Coprococcus, was cultured in vitro to isolate vesicles, and then the vesicles were administered to inflammatory cells in vitro, it was confirmed that the secretion of inflammation mediators, mediated by pathogenic vesicles was significantly inhibited. Therefore, it is expected that the vesicles derived from bacteria of the genus Coprococcus according to the present invention can be effectively used for a method of diagnosing breast cancer, ovarian cancer, bladder cancer, myocardial infarction, cardiomyopathy and atrial fibrillation, and a composition for preventing, treating or alleviating gastritis, gastric cancer, colitis, colon cancer, breast cancer, ovarian cancer, bladder cancer, myocardial infarction, cardiomyopathy, atrial fibrillation or an inflammatory disease.

### DESCRIPTION OF DRAWINGS

FIG. 1A is a series of photographs capturing distribution patterns of bacteria and bacteria-derived vesicles (EV) by time after the bacteria and the vesicles derived from bacteria were orally administered to mice, and FIG. 1B is a result of evaluating the in vivo distribution patterns of the bacteria and the vesicles by harvesting blood, kidneys, liver, and various organs at 12 hours after orally administering the bacteria and the vesicles.

FIG. 2 is a result of comparing the distributions of vesicles derived from bacteria of the genus Coprococcus after metagenomic analysis of bacteria-derived vesicles present in the blood of breast cancer patients and a normal individual.

FIG. 3 is a result of comparing the distributions of vesicles derived from bacteria of the genus Coprococcus after metagenomic analysis of bacteria-derived vesicles present in the blood of ovarian cancer patients and a normal individual.

FIG. 4 is a result of comparing the distributions of vesicles derived from bacteria of the genus Coprococcus after metagenomic analysis of bacteria-derived vesicles present in the blood of bladder cancer patients and a normal individual.

FIG. 5 is a result of comparing the distributions of vesicles derived from bacteria of the genus Coprococcus after metagenomic analysis of bacteria-derived vesicles present in the blood of myocardial infarction patients and a normal individual.

FIG. 6 is a result of comparing the distributions of vesicles derived from bacteria of the genus Coprococcus after metagenomic analysis of bacteria-derived vesicles present in the blood of cardiomyopathy patients and a normal individual.

FIG. 7 is a result of comparing the distributions of vesicles derived from bacteria of the genus Coprococcus after metagenomic analysis of bacteria-derived vesicles present in the blood of atrial fibrillation patients and a normal individual.

FIG. 8 is a result of evaluating an effect on the secretion of IL-6 and TNF-α which inflammatory mediators caused by E. coli EVs by pretreating vesicles derived from Coprococcus comes before treatment of pathogenic vesicles such as E. coli EVs to evaluate anti-inflammatory and immunomodulatory effects of Coprococcus comes-derived vesicles.

### [Best Modes]

The present invention relates to vesicles derived from bacteria of the genus Coprococcus and a use thereof.

The inventors confirmed that vesicles derived from bacteria of the genus Coprococcus are significantly reduced in clinical samples of patients with breast cancer, ovarian cancer, bladder cancer, myocardial infarction, cardiomyopathy and atrial fibrillation, compared with a normal individual, through metagenomic analysis, thereby diagnosing a disease. In addition, as a result of isolating vesicles from Coprococcus comes belonging to bacteria of the genus Coprococcus and analyzing their characteristics, it was confirmed that the vesicles were able to regulate immune reaction by causative factor of inflammation and cancer and can be used for a composition for preventing, treating or alleviating a disease such as gastritis, gastric cancer, colitis, colon cancer, breast cancer, ovarian cancer, bladder cancer, myocardial infarction, cardiomyopathy, atrial fibrillation or an inflammatory disease.

Thus, the present invention provides a method of providing information for diagnosing breast cancer, ovarian cancer, bladder cancer, myocardial infarction, cardiomyopathy or atrial fibrillation, the method comprising the following steps:

(a) extracting DNAs from extracellular vesicles isolated from samples of a normal individual and a subject;

(b) performing polymerase chain reaction (PCR) on the extracted DNA using a pair of primers prepared based on a gene sequence present in 16 S rDNA to obtain each PCR product; and

(c) classifying a case in which a content of vesicles derived from bacteria of the genus Coprococcus is lower than that of the normal individual sample, as breast cancer, ovarian cancer, bladder cancer, myocardial infarction, cardiomyopathy or atrial fibrillation, through quantitative analysis of the PCR product.

The term “diagnosis” as used herein refers to determination of a condition of a disease of a patient over all aspects, in a broad sense. The contents of the determination are the disease entity, the etiology, the pathogenesis, the severity, the detailed aspects of a disease, the presence and absence of complications, the prognosis, and the like. The diagnosis in the present invention means determining whether breast cancer, ovarian cancer, bladder cancer, myocardial infarction, cardiomyopathy and atrial fibrillation occur, the level of the disease, and the like.

The term “nanovesicle” or “vesicle” as used herein refers to a structure consisting of a nano-sized membrane secreted from various bacteria. Vesicles derived from gram-negative bacteria or outer membrane vesicles (OMVs) have endotoxins (lipopolysaccharides), toxic protein, bacterial DNA and RNA, and vesicles derived from gram-positive bacteria also have peptidoglycan and lipoteichoic acid which are cell wall components of bacteria in addition to proteins and nucleic acids. In the present invention, nanovesicles or vesicles are secreted naturally from bacteria of the genus Coprococcus or produced artificially, are in the form of a sphere, and have an average diameter of 10 to 200 nm.

The term “metagenome” as used herein also refers to a microbiome, and refers to a total of genomes including all viruses, bacteria, fungi, and the like in an isolated region such as soil and an animal's intestines, and is typically used as a concept of genomes explaining identification of a large number of microorganisms at one time by using a sequence analyzer in order to analyze uncultivated microorganisms. In particular, the metagenome does not refer to a genome of one species, but refers to a kind of mixed genome as a genome of all species of one environmental unit. The metagenome is, when one species is defined in the development process of omics biology, a term derived from the viewpoint of making a complete species is made by various species interacting with each other as well as one kind of functionally existing species. Technically, the metagenome is an object of a technique to identify all species in one environment and investigate interactions and metabolism by analyzing all DNAs and RNAs regardless of species using a rapid sequence analysis method.

In the present invention, the sample may be blood, but is not limited thereto.

In the present invention, the primer pair in Step (b) may be primers of SEQ ID Nos. 1 and 2.

Another aspect of the present invention provides a composition for preventing or treating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, comprising vesicles derived from bacteria of the genus Coprococcus as an active ingredient. In the present invention, the composition includes a pharmaceutical composition, an oral composition, or an inhalant composition. The composition of the present invention may be prepared in a dosage form of an oral spray or nasal spray.

The term “inflammatory disease” used herein refers to a disease induced by an inflammatory response in a mammalian body, and in the present invention, the inflammatory disease may be one or more selected from the group consisting of atopic dermatitis, acne, psoriasis, sinusitis, rhinitis, conjunctivitis, asthma, dermatitis, an inflammatory collagen vascular disease, glomerulonephritis, encephalitis, inflammatory enteritis, chronic obstructive pulmonary disease, sepsis, septic shock, pulmonary fibrosis, undifferentiated spondylosis, undifferentiated arthrosis, arthritis, inflammatory osteolysis, chronic inflammatory diseases caused by viral or bacterial infections, ulcerative colitis, inflammatory bowel disease, rheumatoid arthritis, reactive arthritis, osteoarthritis, scleroderma, osteoporosis, atherosclerosis, myocarditis, endocarditis, pericarditis, cystic fibrosis, Hashimoto's thyroiditis, Graves' disease, leprosy, syphilis, Lyme disease, borreliosis, neuroborreliosis, tuberculosis, sarcoidosis, lupus, lupus pernio, lupus tuberculosis, lupus nephritis, systemic lupus erythematosus, macular degeneration, uveitis, irritable bowel syndrome, Crohn's disease, Sjogren's syndrome, fibromyalgia, chronic fatigue syndrome, chronic fatigue immunodeficiency syndrome, myalgic encephalomyelitis, amyotrophic lateral sclerosis, Parkinson's disease, and multiple sclerosis, but the present invention is not limited thereto.

The term “prevention” as used herein refers to all actions that suppress gastritis, gastric cancer, colitis, colon cancer, breast cancer, ovarian cancer, bladder cancer, myocardial infarction, cardiomyopathy, atrial fibrillation, an inflammatory disease, or the like or delay the onset thereof via administration of the composition according to the present invention.

The term “treatment” as used herein refers to all actions that alleviate or beneficially change symptoms of gastritis, gastric cancer, colitis, colon cancer, breast cancer, ovarian cancer, bladder cancer, myocardial infarction, cardiomyopathy, atrial fibrillation, an inflammatory disease, or the like via administration of composition according to the present invention.

The term “alleviation” used as used herein refers to all actions that at least reduce a parameter associated with a condition to be treated, for example, the degree of symptoms.

The vesicles may be isolated from a culturing solution comprising bacteria of the genus Coprococcus by using one or more methods selected from the group consisting of centrifugation, ultra-high speed centrifugation, high pressure treatment, extrusion, sonication, cell lysis, homogenization, freezing-thawing, electroporation, mechanical decomposition, chemical treatment, filtration by a filter, gel filtration chromatography, free-flow electrophoresis, and capillary electrophoresis. Further, a process such as washing for removing impurities and concentration of obtained vesicles may be further included.

The pharmaceutical composition of the present invention may include a pharmaceutically acceptable carrier. The pharmaceutically acceptable carrier is typically used in formulation, and includes saline, sterile water, Ringer's solution, buffered saline, cyclodextrin, a dextrose solution, a maltodextrin solution, glycerol, ethanol, liposomes, and the like, but is not limited thereto, and may further include other typical additives such as an antioxidant and a buffer, if necessary. Further, the composition may be formulated into an injectable formulation, such as an aqueous solution, a suspension, and an emulsion, a pill, a capsule, a granule, or a tablet by additionally adding a diluent, a dispersant, a surfactant, a binder, a lubricant, and the like. With regard to suitable pharmaceutically acceptable carriers and formulations, the composition may be preferably formulated according to each ingredient by using the method disclosed in the Remington's literature. The pharmaceutical composition of the present invention is not particularly limited in formulation, but may be formulated into an injection, an inhalant, an external preparation for skin, an oral ingestion, or the like.

The pharmaceutical composition of the present invention may be administered orally or parenterally (e.g., intravenously, subcutaneously, intradermally, intranasally or intratracheally) according to a desired method, and a dose may vary according to the condition and body weight of a patient, the severity of a disease, a drug formulation, an administration route, and duration, but may be suitably selected by those of ordinary skill in the art.

The pharmaceutical composition according to the present invention is administered in a pharmaceutically effective amount. In the present invention, the pharmaceutically effective amount refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields. The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

Specifically, an effective amount of the pharmaceutical composition according to the present invention may vary according to a patient's age, gender and body weight, and generally, the pharmaceutical composition may be administered at 0.001 to 150 mg, and preferably, 0.01 to 100 mg per kg of body weight daily or every two days, or 1 to 3 times daily. However, as the dose may be increased or decreased by an administration route, the severity of obesity, gender, a body weight or an age, the above-mentioned dose does not limit the scope of the present invention in any way.

Another aspect of the present invention provides a food composition for preventing or alleviating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, comprising vesicles derived from bacteria of the genus Coprococcus as an active ingredient.

The food composition of the present invention includes a health functional food composition. The food composition according to the present invention may be used by adding an active ingredient as is to food or may be used together with other foods or food ingredients, but may be appropriately used according to a typical method. The mixed amount of the active ingredient may be suitably determined depending on the purpose of use thereof (for prevention or alleviation). In general, when a food or beverage is prepared, the composition of the present invention is added in an amount of 15 wt % or less, preferably 10 wt % or less based on the raw materials. However, for long-term intake for the purpose of health and hygiene or for the purpose of health control, the amount may be less than the above-mentioned range.

Other ingredients are not particularly limited, except that the food composition of the present invention contains the active ingredient as an essential ingredient at the indicated ratio, and the food composition of the present invention may contain various flavorants, natural carbohydrates, and the like, like a typical beverage, as an additional ingredient. Examples of the above-described natural carbohydrate include common sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavorant other than those described above, a natural flavorant (thaumatin, stevia extract, for example, rebaudioside A, glycyrrhizin and the like), and a synthetic flavorant (saccharin, aspartame and the like) may be advantageously used. The proportion of the natural carbohydrate may be appropriately determined by the choice of those of ordinary skill in the art.

The food composition of the present invention may contain various nutrients, vitamins, minerals (electrolytes), flavoring agents such as synthetic flavoring agents and natural flavoring agents, colorants and fillers (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in a carbonated beverage, or the like, in addition to the additives. These ingredients may be used either alone or in combinations thereof. The ratio of these additives may also be appropriately selected by those of ordinary skill in the art.

In the inhalant composition of the present invention, the active ingredient may be used as it is or used with other ingredients, and may be suitably used according to a conventional method. A mixing content of the active ingredient may be appropriately determined according to its purpose of use (for prevention or treatment).

In the present invention, the inflammatory disease may be a disease mediated by IL-6 or TNF-α, but the present invention is not limited thereto.

Another aspect of the present invention provides a cosmetic composition for preventing or alleviating an inflammatory disease, comprising vesicles derived from bacteria of the genus Coprococcus as an active ingredient.

In the present invention, the cosmetic composition may be used to prevent or alleviate an inflammatory disease selected from the group consisting of atopic dermatitis, acne and psoriasis, but the present invention is not limited thereto.

The cosmetic composition of the present invention may comprise ingredients conventionally used in a cosmetic composition as well as vesicles derived from bacteria of the genus Coprococcus, and may comprise, for example, a conventional additive such as an antioxidant, a stabilizer, a solubilizer, a vitamin, a pigment and a flavor, and a carrier.

In addition, the composition of the present invention may also be used by mixing a conventionally used organic sunscreen as long as it does not impair a skin protection effect by reaction with the vesicles derived from bacteria of the genus Coprococcus, in addition to the vesicles derived from bacteria of the genus Coprococcus. The organic sunscreen may be one or more selected from the group consisting of glyceryl PABA, drometrizole trisiloxane, drometrizole, digalloyl trioleate, disodium phenyl dibenzimidazole tetrasulfonate, diethylhexyl butamidotriazone, diethylamino hydroxybenzoyl hexylbenzoate, DEA-methoxycinnamate, a Lawson/dihydroxyacetone mixture, methylenebis-benzotriazolyltetramethylbutylphenol, 4-methylbenzylidene camphor, methyl anthranilate, benzophenone-3(oxybenzone), benzophenone-4, benzophenone-8(dioxyphebenzone), butyl methoxydibenzoylmethane, bisethylhexyloxyphenol methoxyphenyl triazine, cinoxate, ethyl dihydroxypropyl PABA, octocrylene, ethylhexyldimethyl PABA, ethylhexyl methoxycinnamate, ethylhexyl salicylate, ethylhexyl triazone, isoamyl-p-methoxycinnamate, polysilicon-15 (dimethicodiethylbenzal malonate), terephthalylidene dicamphor sulfonic acid and a salt thereof, TEA-salicylate and aminobenzoic acid (PABA).

Products that can contain the cosmetic composition of the present invention include, for example, cosmetics such as an astringent, a skin toner, a nourishing toner, various types of creams, essences, packs and foundations, cleansers, face washes, soaps, treatments, and tonics. Specific formulations of the cosmetic composition of the present invention include a skin lotion, a skin softener, a skin toner, an astringent, a lotion, a milk lotion, a moisturizing lotion, a nourishing lotion, a massage cream, a nourishing cream, a moisturizing cream, a hand cream, an essence, a nourishing essence, a pack, a soap, a shampoo, a cleansing foam, a cleansing lotion, a cleansing cream, a body lotion, a body cleanser, an emulsion, a lipstick, a makeup base, a foundation, a pressed powder, a loose powder, and an eyeshadow.

In one embodiment of the present invention, as a result of orally administering bacteria and bacteria-derived vesicles to mice and observing in vivo absorption, distribution, and excretion patterns of the bacteria and the vesicles, it was confirmed that, while the bacteria were not absorbed via the intestinal mucous membrane, the bacteria-derived vesicles were absorbed within 5 minutes after administration and systemically distributed, and excreted via the kidneys, liver, and the like (see Example 1).

In another embodiment of the present invention, a bacterial metagenomic analysis was performed by using vesicles isolated from the blood of normal individuals who were matched in age and sex with patients with breast cancer, ovarian cancer, bladder cancer, myocardial infarction, cardiomyopathy and atrial fibrillation. As a result, it was confirmed that vesicles derived from bacteria of the genus Coprococcus were significantly decreased in clinical samples of patients with breast cancer, ovarian cancer, bladder cancer, myocardial infarction, cardiomyopathy and atrial fibrillation as compared to samples of normal individuals (see Examples 3 to 8).

In another embodiment of the present invention, Coprococcus comes was isolated and cultured from gastric fluid to evaluate whether vesicles secreted therefrom have immunomodulatory and anti-inflammatory effects, and it was confirmed that IL-6 and TNF-α secretion caused by Escherichia coli vesicles (E. coli EVs) are effectively inhibited by Coprococcus comes-derived vesicles through evaluation of the secretion of inflammatory mediators by treating E. coli EVs, which are causative factor of an inflammatory disease and cancer, following treatment of macrophages with various concentrations of Coprococcus comes-derived vesicles (see Example 9).

### MODES OF THE INVENTION

Hereinafter, preferred Examples for helping the understanding of the present invention will be suggested. However, the following Examples are provided only to more easily understand the present invention, and the contents of the present invention are not limited by the following Examples.

### EXAMPLES

### Example 1. Analysis of In Vivo Absorption, Distribution, and Excretion Patterns of Intestinal Bacteria and Vesicles Derived from Bacteria

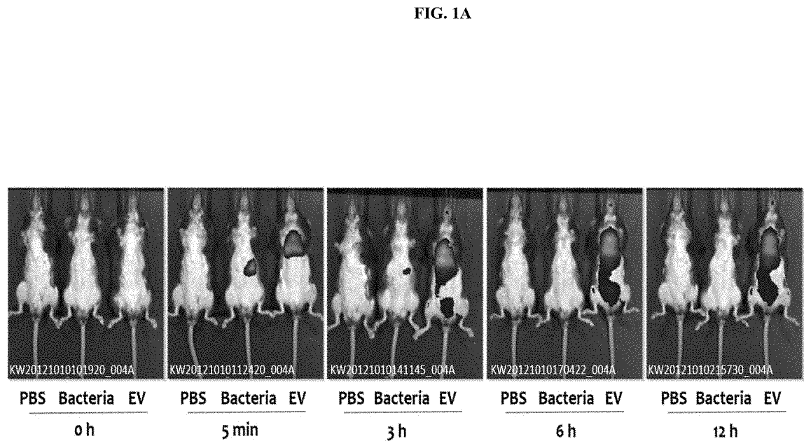

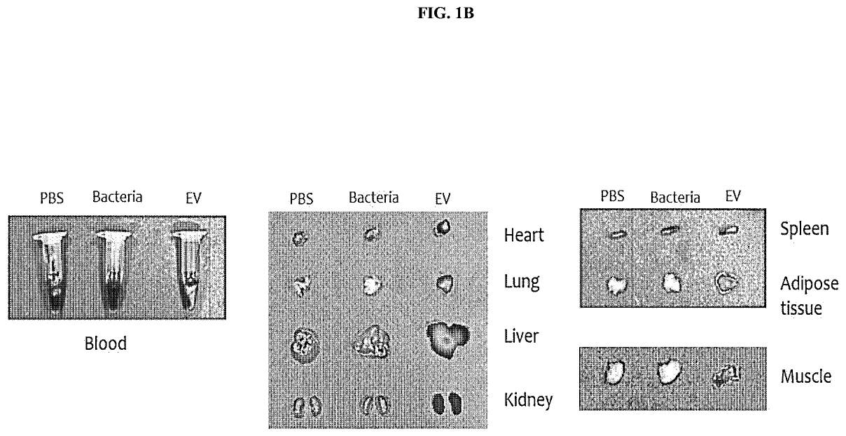

In order to evaluate whether intestinal bacteria and bacteria-derived vesicles were systemically absorbed through the gastrointestinal tract, an experiment was performed with the following method. First, a dose of 50 μg of each of fluorescence-labeled intestinal bacteria and intestinal bacteria-derived vesicles was administered through the gastrointestinal tract to the stomach of a mouse, and fluorescence was measured after 0 minute, 5 minutes, 3 hours, 6 hours, and 12 hours. As a result of observing the entire image of the mouse, as illustrated in FIG. 1A, the bacteria were not systemically absorbed, but the vesicles derived from bacteria were systemically absorbed 5 minutes after administration, and fluorescence was strongly observed in the bladder 3 hours after administration, so that it could be seen that the vesicles were excreted to the urinary tract. Further, it could be seen that the vesicles were present in the body until 12 hours after administration (see FIG. 1A).

In addition, in order to evaluate the pattern in which the intestinal bacteria and the vesicles derived from the intestinal bacteria infiltrated into various organs after they were systemically absorbed, 50 μg of bacteria and vesicles derived from bacteria labeled with fluorescence were administered in the same manner as described above, and then the urine, heart, lungs, liver, kidneys, spleen, fat, and muscle were collected 12 hours after administration. As a result of observing fluorescence in the collected tissues, as illustrated in FIG. 1B, it could be seen that the vesicles derived from bacteria were distributed in the urine, heart, lungs, liver, spleen, fat, muscle, and kidneys but the bacteria were not absorbed (see FIG. 1B).

### Example 2. Metagenomic Analysis of Vesicles Derived from Bacteria in Clinical Sample

After blood was first put into a 10-ml tube and suspended matter was allowed to settle by a centrifuge (3,500×g, 10 min, 4° C.), only the supernatant was transferred to a new 10-ml tube. After bacteria and impurities were removed by using a 0.22-μm filter, they were transferred to a Centriprep tube (centrifugal filters 50 kD) and centrifuged at 1,500×g and 4° C. for 15 minutes, materials smaller than 50 kD were discarded, and the residue was concentrated to 10 ml. After bacteria and impurities were removed once again by using a 0.22-μm filter, the supernatant was discarded by using a ultra-high speed centrifugation at 150,000×g and 4° C. for 3 hours with a Type 90Ti rotor, and an aggregated pellet was dissolved in physiological saline (PBS).

Internal DNA was extracted out of the lipid by boiling 100 μl of the vesicles isolated by the above method at 100° C., and then cooled on ice for 5 minutes. And then, in order to remove the remaining suspended matter, the DNA was centrifuged at 10,000×g and 4° C. for 30 minutes, and only the supernatant was collected. And, the amount of DNA was quantified by using Nanodrop. Thereafter, in order to confirm whether the DNA derived from bacteria was present in the extracted DNA, PCR was performed with 16 s rDNA primers shown in the following Table 1 and it was confirmed that genes derived from bacteria were present in the extracted genes.

TABLE 1

| primer | | Sequence | SEQ ID No. |
|---|---|---|---|
| 16S | 16S_V3_F | 5′-TCGTCGGCAGCGTCAG | 1 |
| rDNA | | ATGTGTATAAGAGACAGC | |
| | | CTACGGGNGGCWGCAG-3′ | |
| | 16S_V4_R | 5′-GTCTCGTGGGCTCGGA | 2 |
| | | GATGTGTATAAGAGACAG | |
| | | GACTACHVGGGTATCTAA | |
| | | TCC | |

The DNA extracted by the above method was amplified using the 16 S rDNA primers, and then sequencing was performed (Illumina MiSeq sequencer), the results were output as a standard flowgram format (SFF) file, the SFF file was converted into a sequence file (.fasta) and a nucleotide quality score file using GS FLX software (v2.9), and then the reliability estimation for the reads was confirmed, and a portion in which the window (20 bps) average base call accuracy was less than 99% (Phred score<20) was removed. For the OTU (operational taxonomy unit) analysis, clustering was performed according to sequence similarity by using UCLUST and USEARCH, the genus, family, order, class, and phylum were clustered based on 94%, 90%, 85%, 80%, and 75% sequence similarity, respectively, classification was performed at the phylum, class, order, family, and genus levels of each OUT, and bacteria having a sequence similarity of 97% or more at the genus level were profiled by using the 16 S RNA sequence database (108,453 sequences) of BLASTN and GreenGenes (QIIME).

### Example 3. Metagenomic Analysis of Bacteria-Derived Vesicles in Blood of Patient with Breast Cancer

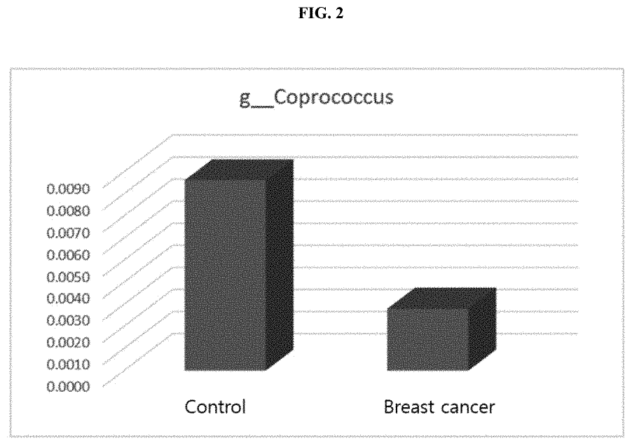

After a metagenomic analysis was performed using the method of Example 2 on the blood from 96 patients with breast cancer, and 192 normal individuals who were matched in age and sex by extracting genes from vesicles present in the blood, the distribution of vesicles derived from bacteria of the genus Coprococcus was evaluated. As a result, it was confirmed that vesicles derived from bacteria of the genus Coprococcus were significantly decreased in the blood from the patients with breast cancer as compared to the blood from the normal individuals (see Table 2 and FIG. 2).

TABLE 2

| Blood | Control | | Breast cancer | | t-test | |
|---|---|---|---|---|---|---|
| Taxon | Mean | SD | Mean | SD | p-value | Ratio |
| g_Coprococcus | 0.0086 | 0.0110 | 0.0028 | 0.0050 | 0.0000 | 0.32 |

### Example 4. Metagenomic Analysis of Bacteria-Derived Vesicles in Blood of Patient with Ovarian Cancer

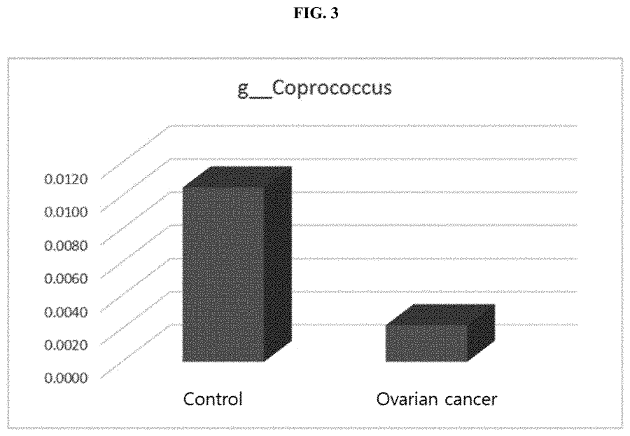

After a metagenomic analysis was performed using the method of Example 2 on the blood from 137 patients with ovarian cancer, and 139 normal individuals who were matched in age and sex by extracting genes from vesicles present in the blood, the distribution of vesicles derived from bacteria of the genus Coprococcus was evaluated. As a result, it was confirmed that vesicles derived from bacteria of the genus Coprococcus were significantly decreased in the blood from the patients with ovarian cancer as compared to the blood from the normal individuals (see Table 3 and FIG. 3).

TABLE 3

| Blood | Control | | Ovarian cancer | | t-test | |
|---|---|---|---|---|---|---|
| Taxon | Mean | SD | Mean | SD | p-value | Ratio |
| g_Coprococcus | 0.0105 | 0.0141 | 0.0022 | 0.0029 | 0.0000 | 0.21 |

### Example 5. Metagenomic Analysis of Bacteria-Derived Vesicles in Blood of Patient with Bladder Cancer

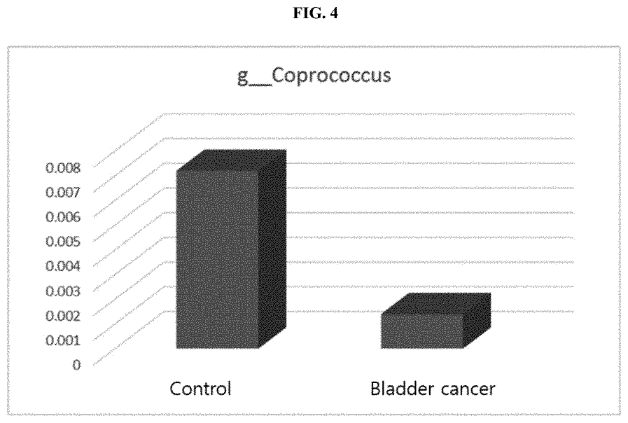

After a metagenomic analysis was performed using the method of Example 2 on the blood from 57 patients with bladder cancer, and 163 normal individuals who were matched in age and sex by extracting genes from vesicles present in the blood, the distribution of vesicles derived from bacteria of the genus Coprococcus was evaluated. As a result, it was confirmed that vesicles derived from bacteria of the genus Coprococcus were significantly decreased in the blood from the patients with bladder cancer as compared to the blood from the normal individuals (see Table 4 and FIG. 4).

TABLE 4

| Blood | Control | | Bbladder cancer | | t-test | |
|---|---|---|---|---|---|---|
| Taxon | Mean | SD | Mean | SD | p-value | Ratio |
| g_Coprococcus | 0.0072 | 0.0077 | 0.0014 | 0.0014 | 0.0000 | 0.20 |

### Example 6. Metagenomic Analysis of Bacteria-Derived Vesicles in Blood of Patient with Myocardial Infarction

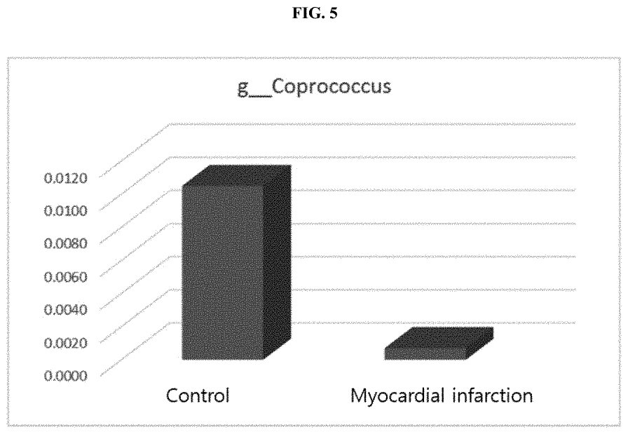

After a metagenomic analysis was performed using the method of Example 2 on the blood from 137 patients with myocardial infarction, and 139 normal individuals who were matched in age and sex by extracting genes from vesicles present in the blood, the distribution of vesicles derived from bacteria of the genus Coprococcus was evaluated. As a result, it was confirmed that vesicles derived from bacteria of the genus Coprococcus were significantly decreased in the blood from the patients with myocardial infarction as compared to the blood from the normal individuals (see Table 5 and FIG. 5).

TABLE 5

| | | | Myocardial | | | |
|---|---|---|---|---|---|---|
| Blood | Control | | infarction | | t-test | |
| Taxon | Mean | SD | Mean | SD | p-value | Ratio |
| g_Coprococcus | 0.0105 | 0.0211 | 0.0007 | 0.0045 | 0.0000 | 0.07 |

### Example 7. Metagenomic Analysis of Bacteria-Derived Vesicles in Blood of Patient with Cardiomyopathy

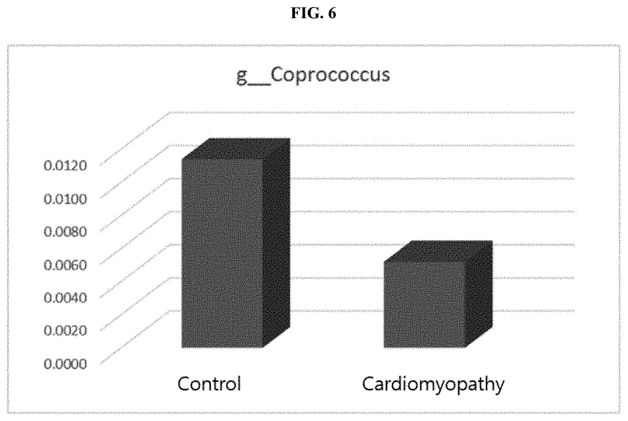

After a metagenomic analysis was performed using the method of Example 2 on the blood from 72 patients with cardiomyopathy, and 163 normal individuals who were matched in age and sex by extracting genes from vesicles present in the blood, the distribution of vesicles derived from bacteria of the genus Coprococcus was evaluated. As a result, it was confirmed that vesicles derived from bacteria of the genus Coprococcus were significantly decreased in the blood from the patients with cardiomyopathy as compared to the blood from the normal individuals (see Table 6 and FIG. 6).

TABLE 6

| Blood | Control | | Cardiomyopathy | | t-test | |
|---|---|---|---|---|---|---|
| Taxon | Mean | SD | Mean | SD | p-value | Ratio |
| g_Coprococcus | 0.0114 | 0.0221 | 0.0052 | 0.0065 | 0.0015 | 0.46 |

### Example 8. Metagenomic Analysis of Bacteria-Derived Vesicles in Blood of Patient with Atrial Fibrillation

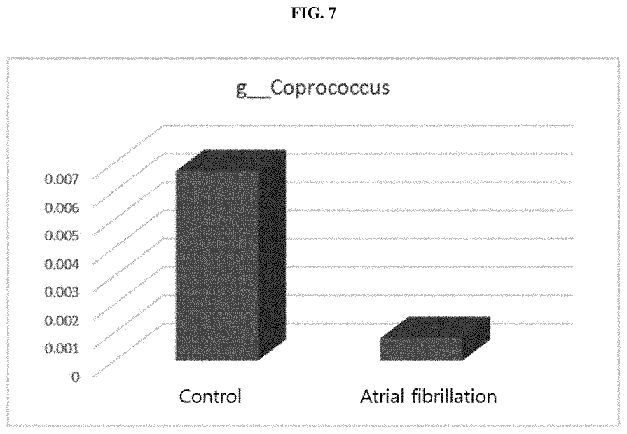

After a metagenomic analysis was performed using the method of Example 2 on the blood from 34 patients with atrial fibrillation, and 62 normal individuals who were matched in age and sex by extracting genes from vesicles present in the blood, the distribution of vesicles derived from bacteria of the genus Coprococcus was evaluated. As a result, it was confirmed that vesicles derived from bacteria of the genus Coprococcus were significantly decreased in the blood from the patients with atrial fibrillation as compared to the blood from the normal individuals (see Table 7 and FIG. 7).

TABLE 7

| Blood | Control | | Atrial fibrillation | | t-test | |
|---|---|---|---|---|---|---|
| Taxon | Mean | SD | Mean | SD | p-value | Ratio |
| g_Coprococcus | 0.0067 | 0.0075 | 0.0008 | 0.0011 | 0.0000 | 0.12 |

### Example 9. Anti-Inflammatory Effects of Coprococcus comes-Derived Vesicles

Based on the result of the above examples, a Coprococcus comes strain belonging to bacteria of the genus Coprococcus was isolated from a gastric fluid and cultured, and vesicles thereof were isolated. The Coprococcus comes strain was cultured in a brain heart infusion (BHI) medium until absorbance (OD600) reached 1.0 to 1.5 in a 37° C. anaerobic chamber and then sub-cultured. Afterward, a medium supernatant which does not contain the strain was collected, centrifuged at 10,000 g and 4 □ for 15 minutes, and filtered through a 0.45-μm filter. A supernatant obtained thereby was concentrated to a volume of 200 mL through ultrafiltration using a QuixStand benchtop system (GE Healthcare, UK) as a 100 kDa hollow filter membrane. Subsequently, the concentrated supernatant was filtered once again with a 0.22-μm filter and ultracentrifuged at 150,000 g and 4 □ for 3 hours, followed by suspension of a pellet in DPBS. Afterward, density gradient centrifugation was performed using 10%, 40% and 50% OptiPrep solutions (Axis-Shield PoC AS, Norway), and to prepare low-density solutions, the OptiPrep solutions were diluted with HEPES-buffered saline (20 mM HEPES, 150 mM NaCl, pH 7.4) before use. After centrifugation for 2 hours under conditions of 200,000 g and 4 □, each solution fractionated with an equal volume of 1 mL from the top layer was additionally ultracentrifuged for 3 hours under conditions of 150,000 g and 4 □. Afterward, a protein was quantified using a bicinchoninic acid (BCA) assay, and an experiment was performed on vesicles obtained as described above.

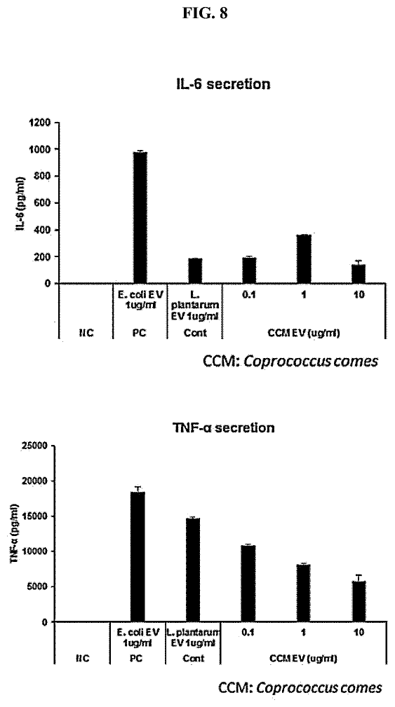

To examine the effect of Coprococcus comes-derived vesicles on the secretion of inflammation mediators in inflammatory cells, Raw 264.7 cells, which is a mouse macrophage cell line, were treated with the Coprococcus comes-derived vesicles at various concentrations (0.1, 1 and 10 μg/mL), and then treated with E. coli EVs, which are pathogenic vesicles of an inflammatory disease, followed by measuring secretion amounts of the inflammation mediators (IL-6 and TNF-α). More specifically, the Raw 264.7 cells were seeded in a 24-well cell culture μlate at 1×10⁵ cells/well, and cultured in complete DMEM for 24 hours. Afterward, a culture supernatant was collected in a 1.5 mL tube and centrifuged at 3000 g for 5 minutes, and then the resulting supernatant was stored at 4 □ and subjected to ELISA analysis. As a result, when Coprococcus comes-derived vesicles were pretreated, it was confirmed that the secretion of the IL-6 and TNF-α by E. coli EVs was significantly suppressed (see FIG. 8). This result shows that inflammatory responses induced by pathogenic vesicles such as E. coli-derived vesicles can be effectively inhibited by the Coprococcus comes-derived vesicles.

The above-described description of the present invention is provided for illustrative purposes, and those of ordinary skill in the art to which the present invention pertains will understand that the present invention can be easily modified into other specific forms without changing the technical spirit or essential features of the present invention. Therefore, it should be understood that the above-described Examples are illustrative only in all aspects and are not restrictive.

### INDUSTRIAL APPLICABILITY

Vesicles derived from bacteria of the genus Coprococcus according to the present invention is expected to be effectively used for a method of diagnosing breast cancer, ovarian cancer, bladder cancer, myocardial infarction, cardiomyopathy or atrial fibrillation, and a food or drug composition for preventing, treating or alleviating gastritis, gastric cancer, colitis, colon cancer, breast cancer, ovarian cancer, bladder cancer, myocardial infarction, cardiomyopathy, atrial fibrillation or an inflammatory disease.

What is claimed is:

1. A method of diagnosing one or more diseases selected from the group consisting of breast cancer, ovarian cancer, bladder cancer, myocardial infarction, cardiomyopathy and atrial fibrillation, the method comprising the following steps:
(a) extracting DNAs from extracellular vesicles isolated from samples of a normal individual and a subject;
(b) performing polymerase chain reaction (PCR) on the extracted DNA using a pair of primers prepared based on a gene sequence present in 16 S rDNA to obtain each PCR product; and
(c) determining a case in which a content of vesicles derived from bacteria of the genus Coprococcus is lower than that of the normal individual sample, as one or more diseases selected from the group consisting of breast cancer, ovarian cancer, bladder cancer, myocardial infarction, cardiomyopathy and atrial fibrillation, through quantitative analysis of the PCR product.

2. The method of claim 1, wherein the sample in Step (a) is blood.

3. A method of treating, preventing, or alleviating one or more diseases selected from the group consisting of gastritis, gastric cancer, colitis, colon cancer, breast cancer, ovarian cancer, bladder cancer, myocardial infarction, cardiomyopathy, atrial fibrillation and an inflammatory disease, the method comprising administering to a subject in need thereof a composition comprising an effective amount of vesicles derived from bacteria of the genus Coprococcus.

4. The method of claim 3, wherein the inflammatory disease is one or more selected from the group consisting of atopic dermatitis, acne, psoriasis, sinusitis, rhinitis, conjunctivitis, asthma, dermatitis, an inflammatory collagen vascular disease, glomerulonephritis, encephalitis, inflammatory enteritis, chronic obstructive pulmonary disease, sepsis, septic shock, pulmonary fibrosis, undifferentiated spondylosis, undifferentiated arthrosis, arthritis, inflammatory osteolysis, chronic inflammatory diseases caused by viral or bacterial infections, ulcerative colitis, inflammatory bowel disease, rheumatoid arthritis, reactive arthritis, osteoarthritis, scleroderma, osteoporosis, atherosclerosis, myocarditis, endocarditis, pericarditis, cystic fibrosis, Hashimoto's thyroiditis, Graves' disease, leprosy, syphilis, Lyme disease, borreliosis, neuroborreliosis, tuberculosis, sarcoidosis, lupus, lupus pernio, lupus tuberculosis, lupus nephritis, systemic lupus erythematosus, macular degeneration, uveitis, irritable bowel syndrome, Crohn's disease, Sjogren's syndrome, fibromyalgia, chronic fatigue syndrome, chronic fatigue immunodeficiency syndrome, myalgic encephalomyelitis, amyotrophic lateral sclerosis, Parkinson's disease, and multiple sclerosis.

5. The method of claim 3, wherein the inflammatory disease is a disease mediated by interleukin-6 (IL-6) or tumor necrosis factor-α (TNF-α).

6. The method of claim 3, wherein the vesicles have an average diameter of 10 to 200 nm.

7. The method of claim 3, wherein the vesicles are secreted naturally or artificially from bacteria of the genus Coprococcus.

8. The method of claim 3, wherein the vesicles derived from bacteria of the genus Coprococcus are vesicles derived from Coprococcus comes.

9. The method of claim 3, wherein the composition is pharmaceutical composition or food composition.

10. The method of claim 3, wherein the composition is an inhalant composition.

11.-15. (canceled)

16. A cosmetic composition for preventing or alleviating inflammatory disease comprising vesicles derived from bacteria of the genus Coprococcus as an active ingredient.

17. The cosmetic composition of claim 16, wherein the inflammatory disease is one or more selected from the group consisting of atopic dermatitis, acne, and psoriasis.

18.-21. (canceled)


SEQUENCE LISTING (2 sequences)

SEQ ID NO: 1 - DNA, 50 bp, Artificial Sequence
  16S_V3_F
  misc_feature (42)..(42): n is a, c, g, or t
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc